(19) United States
(12) Patent Application Publication
(10) Pub. No.: US 2013/0310396 A1
(43) Pub. Date: Nov. 21, 2013

(54) THIAZOLIDINE DERIVATIVES AND THEIR THERAPEUTIC USE

(71) Applicants: Hongwen Zhu, Bronx, NY (US); Qiang Zhang, Somerset, NJ (US)

(72) Inventors: Hongwen Zhu, Bronx, NY (US); Qiang Zhang, Somerset, NJ (US)

(21) Appl. No.: 13/980,788

(22) PCT Filed: Jan. 18, 2012
(86) PCT No.: PCT/US2012/021637

§ 371 Date: Jul. 19, 2013

Related U.S. Application Data
(60) Provisional application No. 61/434,155, filed on Jan. 19, 2011.

(51) Int. Cl. C07D 403/12; A61K 45/06; A61K 31/496

(52) U.S. Cl. CPC C07D 403/12; A61K 31/496; A61K 45/06
USPC 514/254.02; 544/369

(57) ABSTRACT

This invention provides deuterated thiazolidine derivatives and compositions comprising these compounds, which are useful agents for the treatment of hyperglycemia diseases or disorders, in particular diabetes mellitus. The disclosure also provides a method of treating hyperglycemia diseases or disorders, in particular diabetes mellitus, using these deuterated thiazolidine derivatives.

### CROSS-REFERENCE TO RELATED APPLICATIONS

This application claims priority under 35 U.S.C. §119(e) to U.S. Provisional Patent Application Ser. No. 61/434,155, filed on Jan. 19, 2011, which is hereby incorporated by reference in its entirety.

### FIELD OF THE INVENTION

The present invention relates to novel thiazolidine compounds and their stereoisomers, tautomers, prodrugs, and pharmaceutically acceptable salts or solvates, which are effective for treatment of hyperglycemia diseases or conditions, especially diabetes mellitus.

### BACKGROUND OF THE INVENTION

Diabetes mellitus (DM), often referred to as diabetes, is a serious disease worldwide. About 7.8% of Americans suffer from diabetes. Diabetes is diagnosed on the basis of an elevated plasma glucose concentration. It is believed that this disorder is caused by insufficient insulin action. Symptoms of diabetes include polyuria, polydipsia, polyphagia, weight loss, fatigue, frequent infections, tingling/numbness in the hands/feet, and blurred vision etc.

Three major types of diabetes are recognized as Type 1 Diabetes Mellitus (T1DM), Type 2 Diabetes Mellitus (T2DM), and Gestational Diabetes. T1DM results from the body's failure to produce enough insulin. It is estimated that 5-10% of Americans who suffer from diabetes are T1DM. Presently most patients with type I diabetes take insulin replacement therapy. T2DM results from insulin resistance, a condition in which cells fail to use insulin properly, sometimes combined with absolute insulin deficiency. Gestational Diabetes refers to those in pregnant women who have never had diabetes before, but have high blood sugar (glucose) levels during pregnancy. Gestational Diabetes affects about 4% of all pregnant women. Among these three major types of diabetes, T2DM accounts for more than 90% of diabetes mellitus. The biological change that leads to T2DM has not been well understood; however, it is believed that genetic component and lifestyle are main factors to the development of type 2 diabetes.

The current treatment strategies for the treatment of diabetes include: 1) preventing and controlling diabetes through diet, weight control, and exercise; and 2) insulin replacement and oral agents that promote insulin secretion and receptor sensitivity. The goal for the treatment of diabetic patients is to effectively control the levels of blood glucose and to ultimately reduce the complications of diabetes induced by hyperglycemia.

One of the methods to achieve this goal is to target the regulatory enzyme Dipeptidyl Peptidase IV (DPPIV), which plays a catalytic role in the process of signal transduction during immune response leading to T2DM. Inhibition of DPPIV prevents the inactivation of GLP-1, thereby increasing 24-hour levels of GLP-1 both in the fasting and fed states. GLP-1 stimulates insulin secretion, and DPPIV inhibition leads to improved beta cell function. DPPIV inhibition may furthermore result in improved insulin sensitivity. DPPIV inhibitors have a significant advantage over GLP-1 and its analogues, as they are smaller molecules and thus are potentially available by oral administration. At present, DPPIV inhibition has been recognized as a safe and effective treatment option for patients with type 2 diabetes.

Teneligliptin, ((2S,4S)-4-(4-(3-methyl-1-phenyl-1H-pyrazol-5-yl)piperazin-1-yl)pyrrolidin-2-yl)(thiazolidin-3-yl)methanone, is a potent DPPIV inhibitor, a new drug candidate currently in Phase III clinical trials for treatment of T2DM.

Despite the beneficial activities of these medicines, there is a continuing need for the development of new agents that are more effective and have fewer side effects for treating hyperglycemia diseases or conditions, such as diabetes mellitus.

### SUMMARY OF THE INVENTION

The present invention provides novel thiazolidine derivatives for the treatment of diabetes mellitus. In one aspect of the present invention, it provides compounds of formula (I):

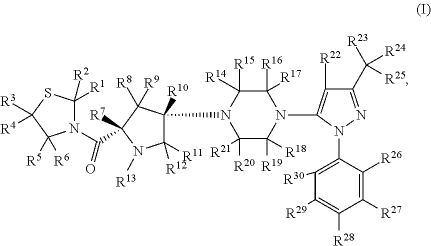

or a stereoisomer, tautomer, prodrug, or pharmaceutically acceptable salt or solvate thereof, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are each independently selected from hydrogen and deuterium, and at least one of R¹, R², R³, R⁴, R⁵, R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ or R³⁰ is deuterium.

The compounds of the present disclosure are effective to inhibit the function of enzyme DPPIV, and are thus useful for the treatment of hyperglycemia, especially for diabetes mellitus. Therefore, this invention also encompasses: (1) compositions comprising a compound of Formula (I), or a stereoisomer, pharmaceutically acceptable salt or solvate thereof, and a pharmaceutically acceptable carrier; (2) a method of treating a hyperglycemia disease or condition, especially diabetes mellitus in a patient, comprising administering to the patient a therapeutically effective amount of a compound of Formula (I), or a pharmaceutically acceptable salt or solvate thereof; and (3) use of a compound of Formula (I) for manufacture of a medicament for treatment of hyperglycemia, especially diabetes mellitus. The diabetes mellitus is more preferably type II diabetes.

The compounds of the present invention are novel, in one aspect, in that one or more natural hydrogens in thiazolidine derivatives are replaced with its non-radioactive isotope, deuterium. This substitution increases the bioavailability, lowers the treatment dosage; and reduces side effects of the drug for treatment of diabetes mellitus.

### DETAILED DESCRIPTION OF THE INVENTION

It is known that Deuterium (D or ²H) is a stable, non-radioactive isotope of hydrogen, with an atomic weight of 2.0144. Hydrogen naturally occurs as a mixture of the isotopes ¹H (hydrogen or protium), D (²H or deuterium), and T (³H or tritium). The concentration of naturally abundant stable hydrogen isotopes is small and immaterial with respect to the degree of stable isotopic substitution of compounds of this invention. (Wada, E. & Hanba, Y., Seikagaku, 1994, 66(1):15-29; Gannes, L. Z. et al., Comp. Biochem. Physiol. A Mol. Integr. Physiol., 1998, 119(3):725-737.) One of ordinary skill in the art recognizes that in all chemical compounds with an H atom, the H atom actually represents a mixture of H and D, with about 0.015% being D. Thus, compounds with a level of deuterium that has been synthesized to be greater than its natural abundance of 0.015% will be considered as unnatural and novel over their natural counterparts.

When a particular position is designated as having deuterium, it is understood that the abundance of deuterium at that position is at least 3400 fold higher than the natural abundance of deuterium (51% deuterium incorporation in the derivative as compared to 0.015% in natural compound).

The present invention, in one aspect, provides novel thiazolidine derivatives for the treatment of hyperglycemia diseases or conditions. In one aspect, the present invention provides compounds of formula (I):

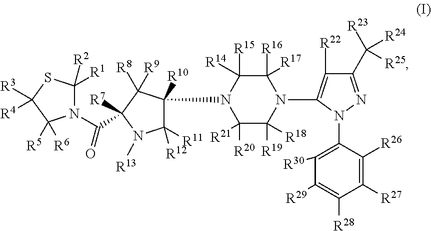

or a stereoisomer, tautomer, prodrug, or pharmaceutically acceptable salt or solvate thereof, wherein:

R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are each independently selected from hydrogen and deuterium; and

at least one of R¹, R², R³, R⁴, R⁵, R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ or R³⁰ is deuterium.

In one embodiment of this aspect, at least one of R¹, R², R³, R⁴, R⁵, or R⁶ is deuterium.

In another embodiment of this aspect, at least one of R⁷, R⁸, R⁹, R¹⁰, R¹¹, or R¹² is deuterium.

In another embodiment of this aspect, at least one of R¹⁴, R¹⁵, R¹⁶, R¹⁷, R¹⁸, R¹⁹, R²⁰, or R²¹ is deuterium.

In another embodiment of this aspect, at least one of R²², R²³, R²⁴, or R²⁵ is deuterium.

In another embodiment of this aspect, at least one of R²⁶, R²⁷, R²⁸, R²⁹ or R³⁰ is deuterium.

In another embodiment of this aspect, the compound of formula (I) is selected from:

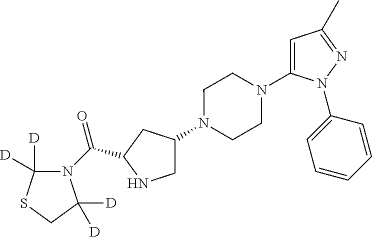

((2S,4S)-4-(4-(3-methyl-1-phenyl-1H-pyrazol-5-yl)piperazin-1-yl)pyrrolidin-2-yl)(2,2,4,4-D₄-thiazolidin-3-yl)methanone;

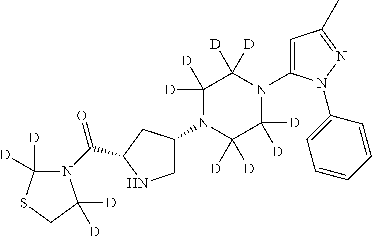 [SMILES: [2H]C1([2H])CSC([2H])([2H])N1C(=O)[C@@H]1C[C@H](N2C([2H])([2H])C([2H])([2H])N(c3cc(C)nn3-c3ccccc3)C([2H])([2H])C2([2H])[2H])CN1]

((2S,4S)-4-(2,2,3,3,5,5,6,6-D₈-4-(3-methyl-1-phenyl-1H-pyrazol-5-yl)piperazin-1-yl)pyrrolidin-2-yl)(2,2,4,4-D₄-thiazolidin-3-yl)methanone;

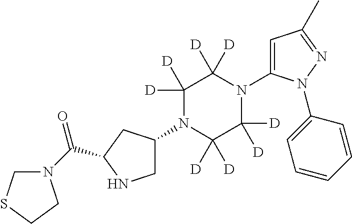 [SMILES: [2H]C1([2H])N(c2cc(C)nn2-c2ccccc2)C([2H])([2H])C([2H])([2H])N([C@@H]2CN[C@H](C(=O)N3CCSC3)C2)C1([2H])[2H]]

((2S,4S)-4-(2,2,3,3,5,5,6,6-D₈-4-(3-methyl-1-phenyl-1H-pyrazol-5-yl)piperazin-1-yl)pyrrolidin-2-yl)(thiazolidin-3-yl)methanone;

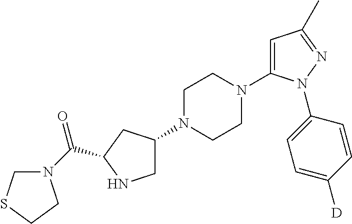 [SMILES: [2H]c1ccc(-n2nc(C)cc2N2CCN([C@@H]3CN[C@H](C(=O)N4CCSC4)C3)CC2)cc1]

((2S,4S)-4-(4-(1-(4-D-phenyl)-3-methyl-1H-pyrazol-5-yl)piperazin-1-yl)pyrrolidin-2-yl)(thiazolidin-3-yl)methanone;

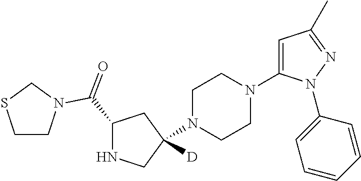 [SMILES: [2H][C@@]1(N2CCN(c3cc(C)nn3-c3ccccc3)CC2)CN[C@H](C(=O)N2CCSC2)C1]

((2S,4S)-4-D-4-(3-methyl-1-phenyl-1H-pyrazol-5-yl)piperazin-1-yl)pyrrolidin-2-yl)(thiazolidin-3-yl)methanone;

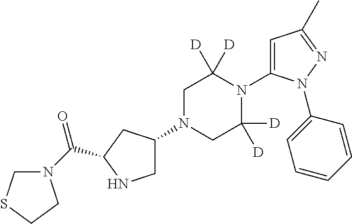 [SMILES: [2H]C1([2H])CN([C@@H]2CN[C@H](C(=O)N3CCSC3)C2)CC([2H])([2H])N1c1cc(C)nn1-c1ccccc1]

((2S,4S)-4-(3,3,5,5-D₄-4-(3-methyl-1-phenyl-1H-pyrazol-5-yl)piperazin-1-yl)pyrrolidin-2-yl)(thiazolidin-3-yl)methanone;

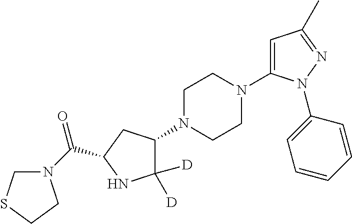 [SMILES: [2H]C1([2H])N[C@H](C(=O)N2CCSC2)C[C@@H]1N1CCN(c2cc(C)nn2-c2ccccc2)CC1]

((2S,4S)-5,5-D₂-4-(4-(3-methyl-1-phenyl-1H-pyrazol-5-yl)piperazin-1-yl)pyrrolidin-2-yl)(thiazolidin-3-yl)methanone;

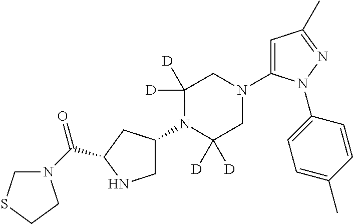 [SMILES: [2H]C1([2H])CN(c2cc(C)nn2-c2ccc(C)cc2)CC([2H])([2H])N1[C@@H]1CN[C@H](C(=O)N2CCSC2)C1]

((2S,4S)-4-(2,2,6,6-D₄-4-(3-methyl-1-phenyl-1H-pyrazol-5-yl)piperazin-1-yl)pyrrolidin-2-yl)(thiazolidin-3-yl)methanone;

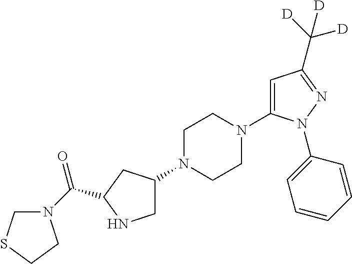

((2S,4S)-4-(4-(3-D₃-methyl-1-phenyl-1H-pyrazol-5-yl)piperazin-1-yl)pyrrolidin-2-yl)(thiazolidin-3-yl)methanone;

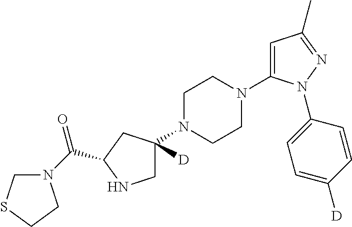

((2S,4S)-4-D-4-(4-(1-(4-D-phenyl)-3-methyl-1H-pyrazol-5-yl)piperazin-1-yl)pyrrolidin-2-yl)(thiazolidin-3-yl)methanone;

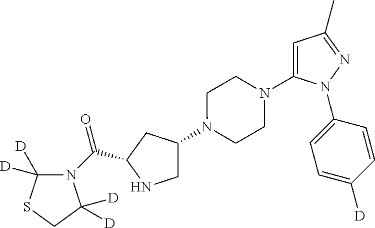 [SMILES: [2H]c1ccc(-n2nc(C)cc2N2CCN([C@@H]3CN[C@H](C(=O)N4C([2H])([2H])CSC4([2H])[2H])C3)CC2)cc1]

((2S,4S)-4-(4-(1-(4-D-phenyl)-3-methyl-1H-pyrazol-5-yl)piperazin-1-yl)pyrrolidin-2-yl)(2,2,4,4-D₄-thiazolidin-3-yl)methanone; and

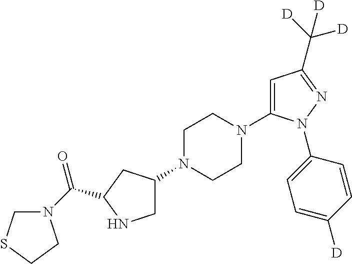

((2S,4S)-4-(4-(3-D₃-methyl-1-(4-D-phenyl)-1H-pyrazol-5-yl)piperazin-1-yl)pyrrolidin-2-yl)(thiazolidin-3-yl)methanone.

In a second aspect, the present disclosure provides a composition comprising a compound of formula (I), or a stereoisomer, tautomer, prodrug, or pharmaceutically acceptable salt or solvate thereof, wherein formula (I) is defined according to any of the embodiments described above.

In one embodiment of this aspect, the composition further comprises a pharmaceutically acceptable carrier.

In some embodiments, the composition further contains one or more additional compounds having anti-hyperglycemia activity. In some embodiments, at least one of the additional compounds is effective to inhibit the activity of enzyme DPPIV.

In a third aspect, the present disclosure provides a method of treating a hyperglycemia disease or disorder in a patient, comprising administering to the patient a therapeutically effective amount of a compound of formula (I), or a stereoisomer, tautomer, prodrug, or pharmaceutically acceptable salt or solvate thereof, wherein formula (I) is defined according to any of the embodiments described in the first aspect of present disclosure. The hyperglycemia disease or disorder is preferably diabetes mellitus, and more preferably type II diabetes.

In one embodiment of the third aspect, the method further comprises administering at least one additional compound having anti-hyperglycemia activity. The administration of the additional compounds can be prior to, after, or simultaneously with administration of the compound of Formula (I), or a pharmaceutically acceptable salt or solvate thereof.

In another embodiment of the third aspect, the present disclosure provides a method of treating a hyperglycemia disease or disorder in a patient, comprising administering to the patient a therapeutically effective amount of a compound of Formula (I), or a stereoisomer, a prodrug, or a pharmaceutically acceptable salt or solvate thereof, in conjunction one or more additional compounds having anti-hyperglycemia activity prior to, after, or simultaneously with the compound of Formula (I), or a pharmaceutically acceptable salt or solvate thereof, wherein at least one of the additional compounds is effective to inhibit the activity of an enzyme DPPIV.

In another aspect the present disclosure provides use of a compound of Formula (I) according to any embodiments defined above for manufacture of a medicament for the treatment of a hyperglycemia disease or disorder.

Other aspects embodiments of the present invention may include any suitable combinations of the embodiments disclosed herein.

The present invention in one aspect is based on the discovery that a therapeutic agent with certain hydrogen atoms replaced by deuterium atoms would possess enhanced stability while maintaining or improving therapeutic potency. Thus, one aspect of this invention is represented by combination of different biological active fragments and/or substitution of natural hydrogen with deuterium to create novel pharmaceutical agents for the treatment of hyperglycemia diseases or disorders, such as diabetes mellitus.

The description of the present disclosure herein should be construed in congruity with the laws and principals of chemical bonding. In some instances it may be necessary to remove a hydrogen atom in order to accommodate a substituent at any given location. Thus, this disclosure is intended to cover all possible stereoisomers

It should be understood that the compounds encompassed by the present disclosure are those that are suitably stable for use as pharmaceutical agents.

In another set of embodiments, any atom not designated as deuterium in any of the embodiments of Formula (I) set forth above is present at its natural isotopic abundance.

Yet other aspects and embodiments may be found in the description or claims provided herein.

### DEFINITIONS

Definitions have been provided above for each of the groups defined. In addition, the following definitions shall be used.

Asymmetric centers exist in the compounds of the present disclosure. These centers are designated by the symbol “R” or “S”, depending on the configuration of substituents around the chiral carbon atom. It should be understood that the disclosure encompasses all stereochemical isomeric forms, or mixtures thereof. Individual stereoisomers of compounds can be prepared synthetically from commercially available starting materials, which contain chiral centers or by preparation of mixtures of enantiomeric products followed by separation, such as conversion to a mixture of diastereomers followed by separation or recrystallization, chromatographic techniques, or direct separation of enantiomers on chiral chromatographic columns. Starting materials of particular stereochemistry are either commercially available or can be made and resolved by techniques known in the art.

In another embodiment of this invention, the compounds of the present invention contain several stereogenic centers. As such, a compound of this invention can exist as the individual stereoisomers (enantiomers or diastereomers) as well as a mixture of stereoisomers.

The compounds of the present disclosure can exist as pharmaceutically acceptable salts or solvates. The term “pharmaceutically acceptable salt,” as used herein, represents salts or zwitterionic forms of the compounds of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use. The salts can be prepared during the final isolation and purification of the compounds or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

In another embodiment of this invention, a salt of the compounds of this invention is formed between an acid and a basic group of the compound, such as an amino functional group, or a base and an acidic group of the compound. The “pharmaceutically acceptable salts,” as used herein, refers to a component which is, within the scope of medical judgment, suitable for use with tissues of humans and other mammals without undesired toxicity, irritation, allergic response or are commensurate with a reasonable benefit/risk ratio. A “pharmaceutically acceptable salt” means any non-toxic salt that, upon administration to a recipient, is capable of providing the compounds or the prodrugs of a compound of this invention.

Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, and N,N′-dibenzylethylenediamine. Other representative organic amines useful for the formation of base addition salts include ethylenediamine, ethanolamine, diethanolamine, triethanolamine, piperidine, piperazine, 1H-imidazole, choline, N-methylglucamine, lysine, arginine, benethamine, benzathine, betaine, decanol, 2-(diethylamini)ethanol, hydrabamine, 4-(2-hydroxyethyl)morpholine, 1-(2-hydroxyethyl)pyrrolidine, and tromethamine.

The term “solvate,” as used herein, means a physical association of a compound of this invention with one or more, preferably one to three, solvent molecules, whether organic or inorganic. This physical association includes hydrogen bonding. In certain instances the solvate will be capable of isolation, for example when one or more, preferably one to three, solvent molecules is incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

The term “prodrug,” as used herein, refers to compounds that are transformed in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable amides of the compounds of this invention include, but are not limited to, primary amides and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

### EXAMPLES

### Synthetic Methods

The compounds of the present invention can be synthesized using the methods described below, together with synthetic methods known in the art of synthetic organic chemistry, or by variations thereon as appreciated by those skilled in the art. It will be understood by those skilled in the art of organic synthesis that the functionality present on the molecule should be consistent with the transformations proposed. This will sometimes requires a judgment to modify the order of the synthetic steps or to select one particular process scheme over another in order to obtain a desired compound of the invention.

Preferred methods include, but are not limited to, those described, for instance, in WO02/14271 and US 2009/0216016A1. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

Common abbreviations listed below may be used in this disclosure.

Me methyl

Pr propyl

i-Pr isopropyl

Bu butyl

t-Bu tert-butyl

Ph phenyl

Boc tert-butyloxycarbonyl

(Boc)₂O di-tert-butyl dicarbonate

AcOH or HOAc acetic acid

CH₂Cl₂ dichloromethane

CH₃CN or ACN acetonitrile

D₂ deuterium gas

DCC N,N′-dicyclohexylcarbodiimide

DCM dichloromethane

DMF dimethyl formamide

Et₃N or TEA triethylamine

EtOAc ethyl acetate

Et₂O diethyl ether

EtOH ethanol

HCl hydrochloric acid

HNO₃ nitric acid

H₂SO₄ sulfuric acid

K₂CO₃ potassium carbonate

K₃PO₄ potassium phosphate

LiAlD₄ lithium aluminum hydride-d₄

LiOH lithium hydroxide

MeOH methanol

MgSO₄ magnesium sulfate

MsOH or MSA methylsulfonic acid

NaBD₄ sodium borohydride-d₄

NaCl sodium chloride

NaHCO₃ sodium bicarbonate

Na₂CO₃ sodium carbonate

NaOH sodium hydroxide

NaH sodium hydride

Na₂S₂O₅ sodium metadisulfite

Na₂SO₄ sodium sulfate

NH₃ ammonia

NH₄Cl ammonium chloride

NH₄OH ammonium hydroxide

Pd(OAc)₂ palladium(II) acetate

Pd/C palladium on carbon

POCl₃ phosphorus oxychloride

SnCl₂ tin chloride

TFA trifluoroacetic acid

THF tetrahydrofuran

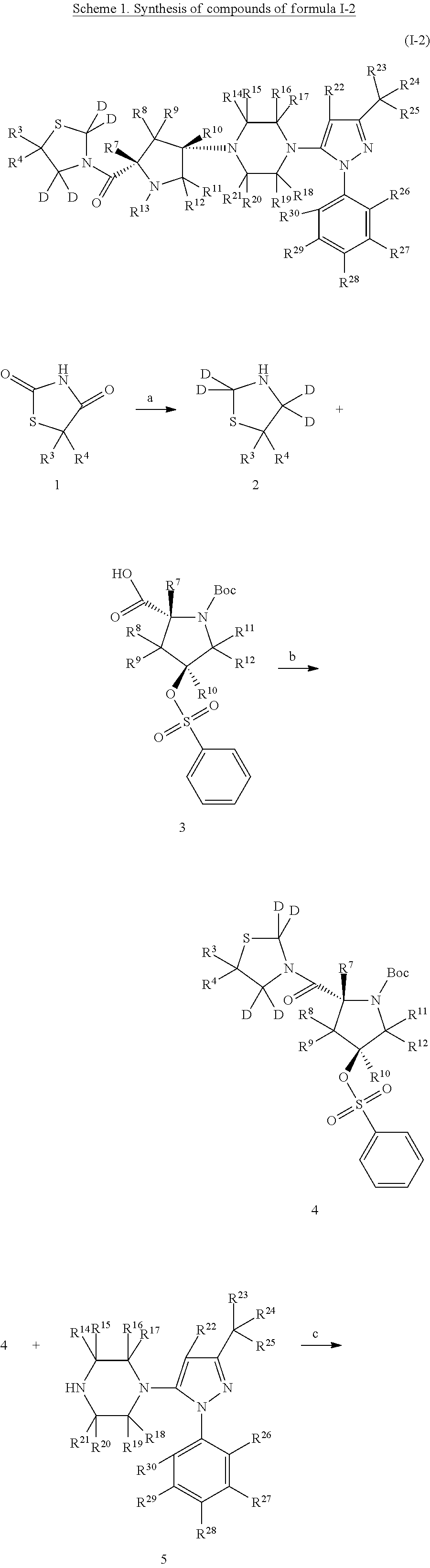

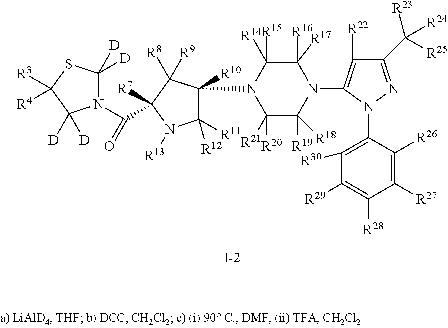

A convenient method for the synthesis of compounds of formula (I-2) is depicted in Scheme 1. As shown in Scheme 1, intermediates 2 are synthesized from commercially available starting material 1 with LiAlD₄. 3 can be synthesized from D-proline analogs with selective protection of amino groups with Boc and protection of hydroxyl groups with benzenesulfonyl esters. After a coupling reaction between 2 and 3, the benzenesulfonyl esters of 4 are further substituted with amines 5 to finish the synthesis of compounds of formula I-2. The intermediates 5 are steadily synthesized form commercially available 9 with amines 8 under classic metal mediated coupling reaction conditions.

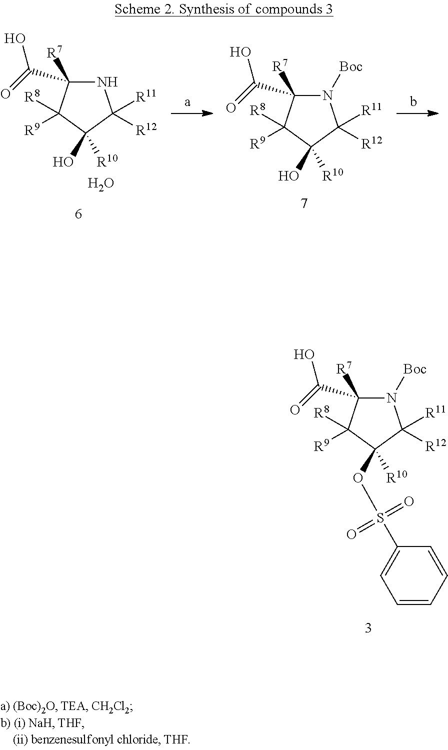

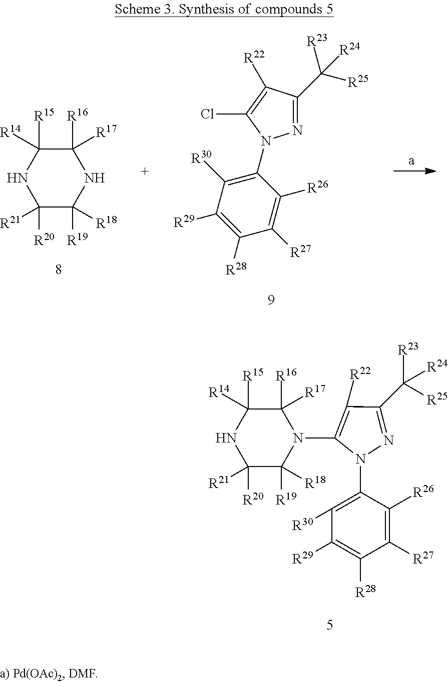

A convenient method for the synthesis of compounds with formula I-3 is depicted in Scheme 4. The deuterated phenylhydrazine is prepared from p-chloronitrobenzene by dehalogenation with deuterium gas and a diazonation reaction followed by a selective reduction. Following the chemistry for the synthesis of 5, intermediates 12 are synthesized in two steps: first, by refluxing mixture of deuterated phenylhydrazine 10 and acetylethylacetaes 11, which gives intermediate 1H-pyrazol-5-ones in high yields; then converting intermediate 1H-pyrazol-5-ones into 12 by refluxing in POCl₃. Intermediates 13 can be readily synthesized from 12 and piperazines 8 under classic metal mediated coupling reaction conditions.

Using the procedure to prepare 4, intermediates 15 can be efficiently synthesized by a coupling reaction between 14 and 3. The synthetic procedure is shown in Scheme 4. Benzenesulfonyl esters of 15 can be further substituted with amines 13. After cleaving Boc protecting groups, compounds of formula I-3 are obtained.

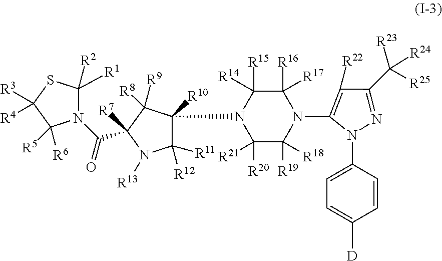

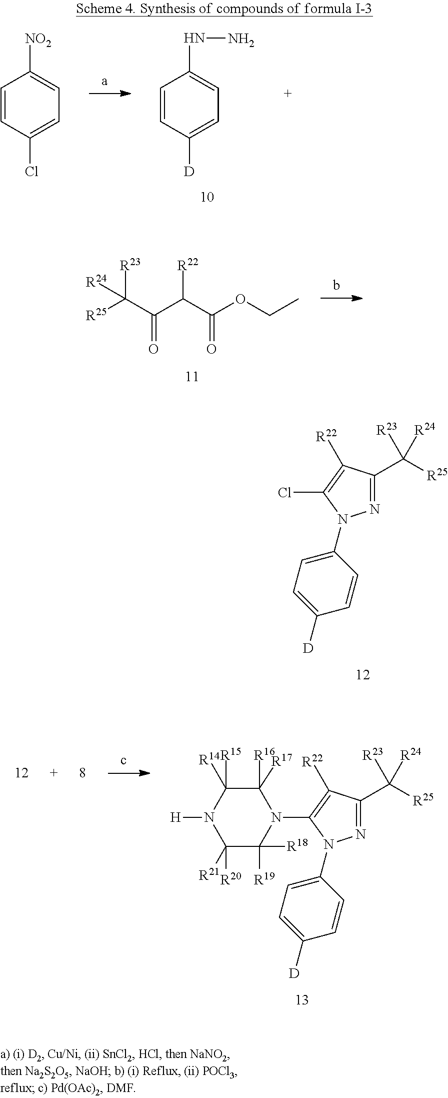

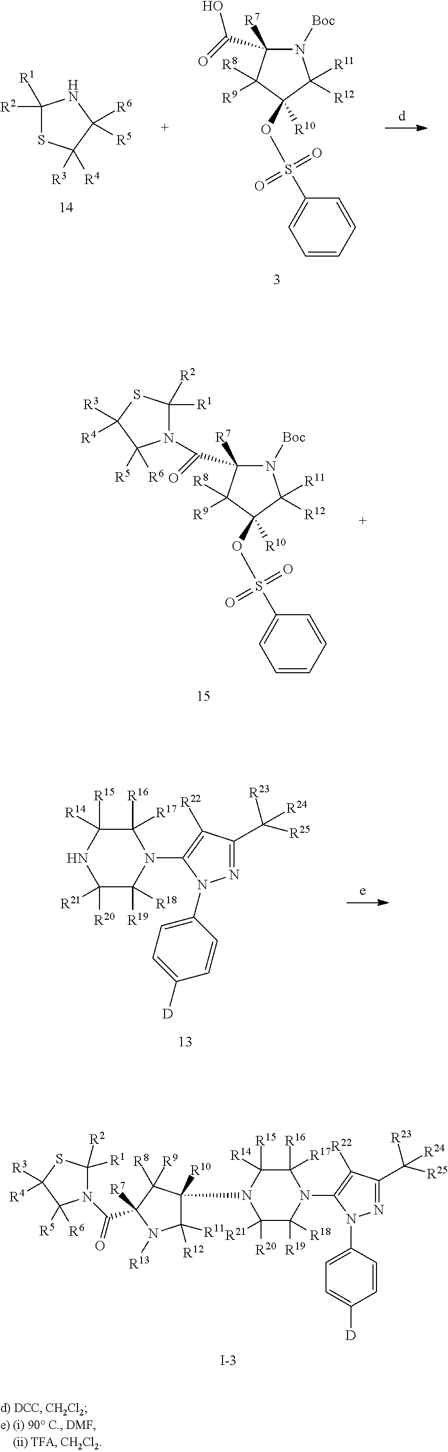

Certain preferred embodiments of the present invention are illustrated in the following non-limiting examples.

### Example 1

### ((2S,4S)-4-(4-(3-Methyl-1-phenyl-1H-pyrazol-5-yl)piperazin-1-yl)pyrrolidin-2-yl)(2,2,4,4-D₄-thiazolidin-3-yl)methanone

### 2,2,4,4-D₄-Thiazolidine (2)

Thiazolidine-2,4-dione (1) (1.2 gram, 10 mmol) was dissolved in 100 ml THF and cooled to 0° C. While stirring, 1.2 equivalent of LiAlD₄ was added in small portions and the mixture was warmed gradually to room temperature in 30 min. The mixture was stirred for 2 h, and solvent was concentrated. The residue was carefully dissolved in dichloromethane and extracted by water. The organic layer was dried and concentrated. The crude product 2 was used directly for the next step. HPLC-MS: m/z 93.4 (M+1)⁺.

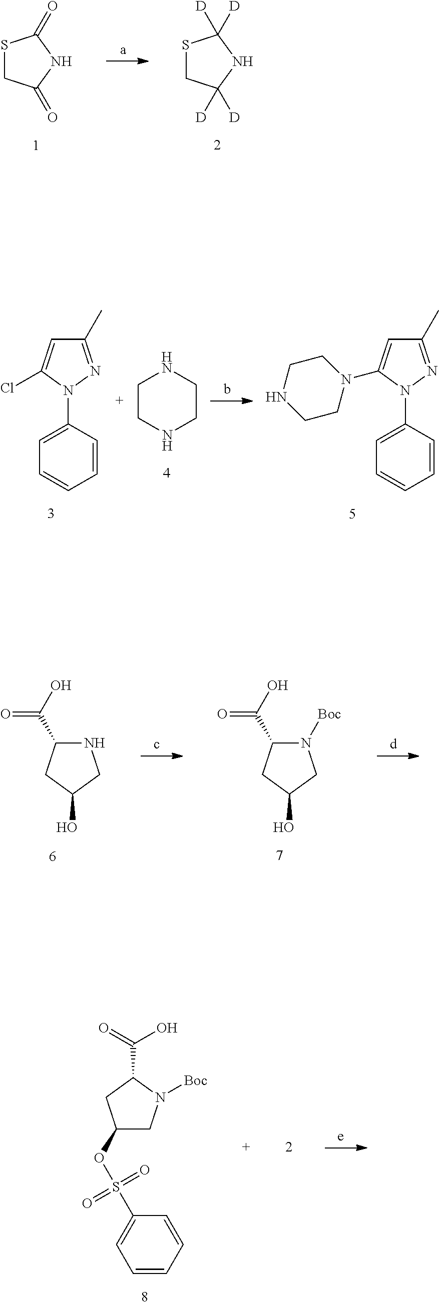

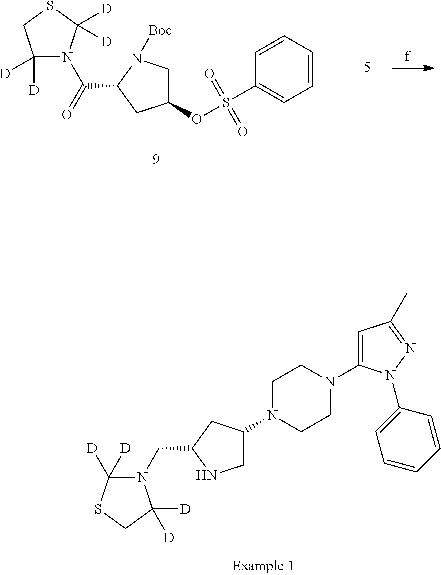

### 1-(3-Methyl-1-phenyl-1H-pyrazol-5-yl)piperazine (5)

5-Chloro-3-methyl-1-phenyl-1H-pyrazole (3) (195 mg, 1.0 mmol) and 1.2 equivalents of piperazine were dissolved in 20 ml DMF. While stirring, 210 mg K₂CO₃ and 225 mg Pd(OAc)₂ were added in sequence. The mixture was purged with nitrogen and heated to 110° C. for 12 h. After cooling to room temperature, the solid was filtrated, and solvent was evaporated. The product was purified by column chromatography to give 180 mg of product 5 as a pale oil, yield 75%.

### (2R,4S)-1-(tent-Butoxycarbonyl)-4-(phenylsulfonyloxy)pyrrolidine-2-carboxylic acid (8)

Compound 6 (135 mg, 1.0 mmol) was dissolved in 20 ml THF, which contained 1.2 equivalents of triethylamine. While stirring, 265 mg of (Boc)₂O (1.2 mmol) was added, and the mixture was stirred for an additional 8 h. Solvent was evaporated, and residue was dissolved in 100 ml THF, and 1.2 mmol of NaH was added at 0° C. After stirring at 0° C. for 30 min, benzenesulfonyl chloride (212 mg, 1.2 mmol) was added in small portions. The mixture was stirred overnight at room temperature. The solvent was concentrated, and the residue was purified by column chromatography to give 225 mg of compound 8, yield 60%. HPLC-MS: m/z 370(M−1)⁻.

### (2R,4S)-tert-Butyl-4-(phenylsulfonyloxy)-2-(2,2,4,4-D4-thiazolidine-3-carbonyl)pyrrolidine-1-carboxylate (9)

Compound 8 (200 mg, 0.54 mmol) was dissolved in 50 ml dichloromethane, and 1.2 equivalents of N,N′-dicyclohexylcarbodiimide (DCC) was added. The solution was stirred for 0.5 h, and 55 mg intermediate 2 was added. The mixture was stirred at room temperature overnight. The product was purified by column chromatography to give 215 mg of compound 9 as a light yellow solid, yield 90%.

### ((2S,4S)-4-(4-(3-Methyl-1-phenyl-1H-pyrazol-5-yl)piperazin-1-yl)pyrrolidin-2-yl)-(2,2,4,4-D₄-thiazolidin-3-yl)methanone (Example 1)

Purified compound 9 (100 mg, 0.23 mmol) and 0.23 mmol intermediate 5 were added in 25 ml DMF. While stirring, 48 mg K₂CO₃ (0.35 mmol) was added and the mixture was stirred at 90° C. overnight. The solvent was evaporated, and the residue was dissolved in a mixture of 5 ml dichloromethane and 5 ml trifluoroacetic acid. After stirring at room temperature for 2 h, the solvents were evaporated, and the residue was purified by column chromatography to give 71 mg product (Example 1) as a white solid, yield 72%, HPLC-MS: m/z 431(M+1)⁺.

### Example 4

### ((2S,4S)-4-(4-(1-(4-D-Phenyl)-3-methyl-1H-pyrazol-5-yl)piperazin-1-yl)pyrrolidin-2-yl)(thiazolidin-3-yl)methanone

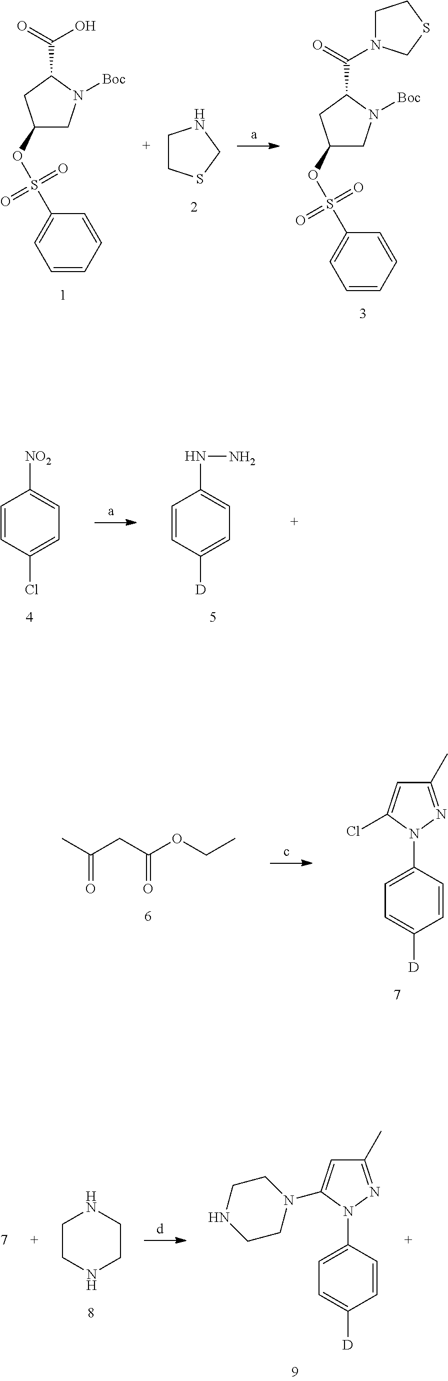

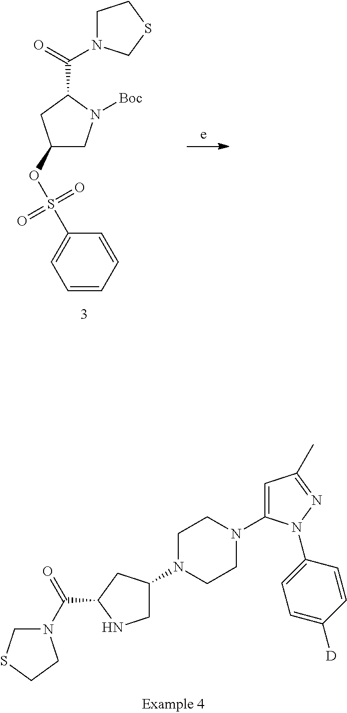 [SMILES: CC(C)(C)OC(=O)N1C[C@@H](OS(=O)(=O)c2ccccc2)C[C@@H]1C(=O)N1CCSC1 ; [2H]c1ccc(-n2nc(C)cc2N2CCN([C@@H]3CN[C@H](C(=O)N4CCSC4)C3)CC2)cc1]

### (2R,4S)-tert-Butyl-4-(phenylsulfonyloxy)-2-(thiazolidine-3-carbonyl)pyrrolidine-1-carboxylate (3)

Starting material 1 (500 mg), which was prepared as shown in the synthesis of example 1, was dissolved in 50 ml dichloromethane, and 1.2 equivalents of DCC was added. The solution was stirred for 1.0 h, and 132 mg thiazolidine (1.48 mmol) was added, and the mixture was stirred at room temperature overnight. The product was purified by column chromatography to give 210 mg compound 3 as a light yellow solid, yield 90%.

### 4-D-Phenylhydrazine (5)

p-Nitro-chlorobenzene (3.14 gram, 20 mmol) was dissolved in 100 ml acetonitrile, and 300 mg Cu/Ni catalyst was added. The mixture was degassed by deuterium gas and stirred at room temperature for 2 h to give 4-D-nitrobenzene. The solid was filtrated, and 2.0 gram of SnCl₂ was added. After stirring at room temperature overnight, the solid was filtrated, and the solvent was evaporated under vacuum. The residue was dissolved in 100 ml 1N HCl, and 800 mg NaNO₂ (20 mmol) was added in small portions. TLC showed that the diazonation reaction finished within 2 h. The acid solution was basified with 10N NaOH and extracted by dichloromethane. The dichloromethane solution was dried and concentrated, and the residue was suspended in 100 ml 1N NaOH solution, which contained 2.0 gram Na₂S₂O₅. The mixture was vigorously stirred overnight and extracted by dichloromethane. The organic layer was dried and concentrated. The product was purified by column chromatography to give compound 5 as a pale oil, yield 40%.

### 5-Chloro-1-(4-D-phenyl)-3-methyl-1H-pyrazole (7)

Compound 5 (500 mg, 4.58 mmol) was dissolved in 10 ml ethyl acetylacetate (6), and the solution was heated to reflux overnight. The excess 6 was evaporated, and the residue was dissolved in 50 ml POCl₃. The solution was heated to reflux for 3 h and concentered. The crude product was purified by column chromatography to give 730 mg compound 7 as a white solid, yield 82%. HPLC-MS: m/z 194(M+1)⁺.

### 1-(1-(4-D-Phenyl)-3-methyl-1H-pyrazol-5-yl)piperazine (9)

Purified compound 7 (200 mg, 1.03 mmol) was dissolved in 20 ml DMF, and 1.2 equivalents of piperazine were dissolved in 20 ml DMF. While stirring, 220 mg K₂CO₃ and 225 mg Pd(OAc)₂ were added in sequence. The mixture was purged by nitrogen and heated to 110° C. for 12 h. After cooling to room temperature, the solid was filtrated and solvent was evaporated. The product was purified by column chromatography to give 200 mg compound 9 as a pale solid, yield 81%. HPLC-MS: m/z 244(M+1)⁺.

### ((2S,4S)-4-(4-(1-(4-D-Phenyl)-3-methyl-1H-pyrazol-5-yl)piperazin-1-yl)pyrrolidin-2-yl)(thiazolidin-3-yl)methanone (Example 4)

Purified compound 9 (100 mg, 0.41 mmol) and 0.40 mmol compound 3 were added in 25 ml DMF. While stirring, 85 mg K₂CO₃ (0.62 mmol) was added and the mixture was stirred at 90° C. overnight. The solvent was evaporated, and the residue was dissolved in 5 ml dichloromethane and 5 ml trifluoroacetic acid. After stirring for 2 h at room temperature, the solvents were evaporated and residue was purified by column chromatography to give 122 mg product (Example 4) as a white solid, yield 72%, HPLC-MS: m/z 428(M+1)⁺.

### Example 5

### ((2S,4S)-4-D-4-(3-Methyl-1-phenyl-1H-pyrazol-5-yl)piperazin-1-yl)pyrrolidin-2-yl)(thiazolidin-3-yl)methanone

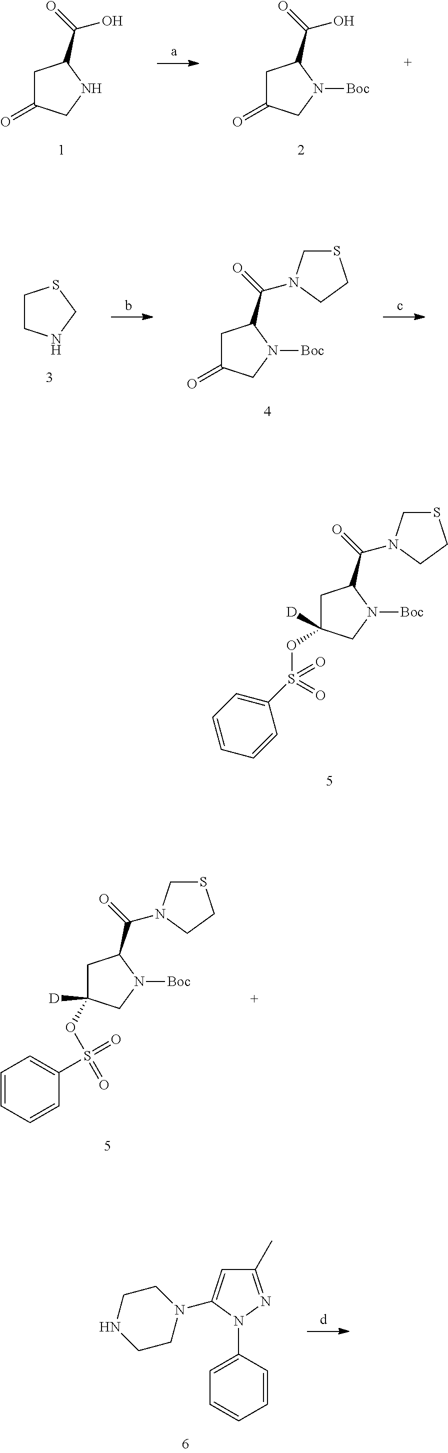

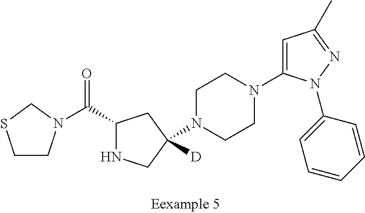 [SMILES: [2H][C@@]1(N2CCN(c3cc(C)nn3-c3ccccc3)CC2)CN[C@H](C(=O)N2CCSC2)C1]

### (S)-tert-Butyl 4-oxo-2-(thiazolidine-3-carbonyl)pyrrolidine-1-carboxylate (4)

(S)-4-Oxopyrrolidine-2-carboxylic acid (1) (1.3 gram, 10 mmol) was dissolved in 100 ml THF, which contained 1.2 equivalent of triethylamine. While stirring, 2.6 gram (Boc)₂O (12 mmol) was added, and the mixture was stirred for an additional 8 h. The solvent was evaporated, and the residue was dissolved in dichloromethane. The organic solution was extracted by water, 1N HCl and brine. The crude product 2 was used directly for the next step.

Compound 2 (500 mg, 2.0 mmol) was dissolved in 100 ml dichloromethane, and 1.2 equivalent of DCC was added. The solution was stirred for 1.0 h, 200 mg thiazolidine (2.2 mmol) was added, and the mixture was stirred at room temperature overnight. The product was purified by column chromatography to give 550 mg compound 4 as a light yellow solid, yield 91%.

### (2S,4S)-tert-Butyl 4-D-4-(phenylsulfonyloxy)-2-(thiazolidine-3-carbonyl)pyrrolidine-1-carboxylate (5)

Purified compound 4 (250 mg, 0.83 mmol) was dissolved in 20 ml acetonitrile, and NaBD₄ (35 mg, 0.84 mmol) was added slowly. The mixture was stirred at room temperature for 2 h. Water (2 ml) was carefully dropped in under stirring and the mixture was extracted by dichloromethane. The organic solution was dried, the solvent was evaporated, and the residue was dissolved in 100 ml dry THF. The solution was cooled to 0° C. and 40 mg NaH was added. After stirring for 30 min at 0° C., benzenesulfonyl chloride (175 mg, 1.0 mmol) was added in small portions. The mixture was stirred at room temperature overnight. The solvent was concentrated and residue was purified by column chromatography to give 300 mg of racemic product, yield 82%. HPLC-MS: m/z 443(M+1)⁺. The racemic compound was separated on Chiral OD column to give 130 mg of the desired product 5 as a white solid.

### ((2S,4S)-4-D-4-(3-Methyl-1-phenyl-1H-pyrazol-5-yl)piperazin-1-yl)pyrrolidin-2-yl)(thiazolidin-3-yl)methanone (Example 5)

Purified compound 6 (100 mg, 0.41 mmol) and compound 5 (175 mg, 0.40 mmol) were added in 25 ml DMF. Under stirring, K₂CO₃ (85 mg, 0.62 mmol) was added, and the mixture was stirred at 90° C. overnight. The solvent was evaporated and residue was dissolved in 5 ml dichloromethane and 5 ml trifluoroacetic acid. After stirring for 2 h at room temperature, the solvents were evaporated, and the residue was purified by column chromatography to give 130 mg product as a white solid, yield 76%, HPLC-MS: m/z 428(M+1)⁺.

### Evaluation of Compound Stability in Human Liver Microsomes

The liver microsomes stability of compounds of Examples 1, 4 and 5 were compared with Teneligliptin.

### Assay System

The metabolic stability of compounds of the invention was tested using pooled liver microsomes prepared from mixed-gender humans, with 1 mM NADPH. The samples were analyzed using an LTQ-Orbitrap XL mass spectrometer. HRMS was used to determine the peak area response ratio (peak area corresponding to test compound or control divided by that of an analytical internal standard) without running a standard curve. HRMS scan was performed in an appropriate m/z range in order to detect all plausible metabolites.

### Assay Conditions

The assay was run with a single incubation (N=1). Incubated test compounds at 37° C. in a buffer solution containing 0.5 mg/mL microsomal protein. Initiated the reaction by adding cofactors, sampling at 0, 10, 20, 30, and 60 minutes. Incubated positive control (5 μM testosterone) in parallel and sampling at 0, 5 10, and 30 minutes.

### Assay QC

The control compound testosterone was run in parallel to verify the enzymatic activity of the microsomes. After the final time point, fluorimetry was used to confirm the addition of NADPH to the reaction mixture. T_{1/2} of control met the internal acceptance criteria.

### Analytical Method

### Liquid Chromatography

Column: Thermo BDS Hypersil C₁₈ 30×2.0 mm, 3 μm, with guard column
M.P. Buffer: 25 mM ammonium formate buffer, pH 3.5
Aqueous Reservoir (A): 90% water, 10% buffer
Organic Reservoir (B): 90% acetonitrile, 10% buffer

### Flow Rate: 300 μL/minute

### Gradient Program:

| Time (Min) | % A | % B |
|---|---|---|
| 0.0 | 100 | 0 |
| 1.5 | 0 | 100 |
| 2.0 | 0 | 100 |
| 2.1 | 100 | 0 |
| 3.5 | 100 | 0 |

Total Run Time: 3.5 minutes

### Autosampler: 10 μL Injection Volume

Autosampler Wash: water/methanol/2-propanol: 1/1/1; with 0.2% formic acid

### Mass Spectrometer

### Instrument: PE SCIEX API 3000

### Interface: Turbo Ionspray

### Mode: Multiple Reaction Monitoring

Method: 3.5 minute duration

TABLE 1
Stability in Human Liver Microsomes

| Test | Percentage Remaining | | | | | Half Life |
|---|---|---|---|---|---|---|
| Compound | 0 min | 10 min | 20 min | 30 min | 60 min | (min) |
| Example 1 | 100 | 92 | 85 | 70 | 60 | >60 |
| Example 4 | 100 | 95 | 87 | 75 | 65 | >60 |
| Example 5 | 100 | 90 | 87 | 70 | 60 | >60 |
| Teneligliptin | 100 | 90 | 85 | 65 | 55 | >60 |

The results are shown in Table 1. Comparing with Teneligliptin, all compounds as exemplified in the table dramatically improve their human liver microsome stabilities. Therefore, they can potentially lower medical dosage comparing with the reference compounds.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein. All patents, patent applications, and literature references cited in the specification are herein incorporated by reference in their entirety. In the case of inconsistencies, the present disclosure, including definitions, will prevail.

1. A compound of formula (I): 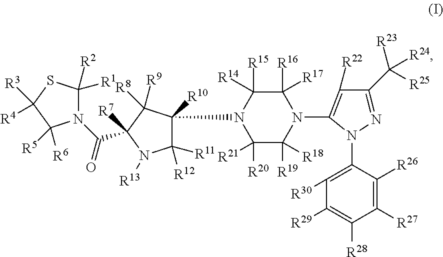
or a stereoisomer, tautomer, prodrug, or pharmaceutically acceptable salt or solvate thereof, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ and R³⁰ are each independently, selected from hydrogen and deuterium; and at least one of R¹, R², R³, R⁴, R⁵, R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹ or R³⁰ is deuterium.

2. The compound of claim 1, or a stereoisomer, tautomer, prodrug, or pharmaceutically acceptable salt or solvate thereof, wherein at least one of R¹, R², R³, R⁴, R⁵, or R⁶ is deuterium.

3. The compound of claim 1, or a stereoisomer, tautomer, prodrug, or pharmaceutically acceptable salt or solvate thereof, wherein at least one of R⁷, R⁸, R⁹, R¹⁰, R¹¹, or R¹² is deuterium.

4. The compound of claim 1, or a stereoisomer, tautomer, prodrug, or pharmaceutically acceptable salt or solvate thereof, wherein at least one of R¹⁴, R¹⁵, R¹⁶, R¹⁷, R¹⁸, R¹⁹, R²⁰, or R²¹ is deuterium.

5. The compound of claim 1, or a stereoisomer, tautomer, prodrug, or pharmaceutically acceptable salt or solvate thereof, wherein at least one of R²², R²³, R²⁴, or R²⁵ is deuterium.

6. The compound of claim 1, or a stereoisomer, tautomer, prodrug, or pharmaceutically acceptable salt or solvate thereof, wherein at least one of R²⁶, R²⁷, R²⁸, R²⁹ or R³⁰ is deuterium.

7. The compound of claim 1, or a stereoisomer, tautomer, prodrug, or pharmaceutically acceptable salt or solvate thereof, selected from the group consisting of:
((2S,4S)-4-(4-(3-methyl-1-phenyl-1H-pyrazol-5-yl)piperazin-1-yl)pyrrolidin-2-yl)(2,2,4,4-D₄-thiazolidin-3-yl)methanone;
((2S,4S)-4-(2,2,3,3,5,5,6,6-D₈-4-(3-methyl-1-phenyl-1H-pyrazol-5-yl)piperazin-1-yl)pyrrolidin-2-yl)(2,2,4,4-D₄-thiazolidin-3-yl)methanone;
((2S,4S)-4-(2,2,3,3,5,5,6,6-D₈-4-(3-methyl-1-phenyl-1H-pyrazol-5-yl)piperazin-1-yl)pyrrolidin-2-yl)(thiazolidin-3-yl)methanone;
((2S,4S)-4-(4-(1-(4-D-phenyl)-3-methyl-1H-pyrazol-5-yl)piperazin-1-yl)pyrrolidin-2-yl)(thiazolidin-3-yl)methanone;
((2S,4S)-4-D-4-(3-methyl-1-phenyl-1H-pyrazol-5-yl)piperazin-1-yl)pyrrolidin-2-yl)(thiazolidin-3-yl)methanone;
((2S,4S)-4-(3,3,5,5-D₄-4-(3-methyl-1-phenyl-1H-pyrazol-5-yl)piperazin-1-yl)pyrrolidin-2-yl)(thiazolidin-3-yl)methanone;
((2S,4S)-5,5-D₂-4-(4-(3-methyl-1-phenyl-1H-pyrazol-5-yl)piperazin-1-yl)pyrrolidin-2-yl)(thiazolidin-3-yl)methanone;
((2S,4S)-4-(2,2,6,6-D₄-4-(3-methyl-1-phenyl-1H-pyrazol-5-yl)piperazin-1-yl)pyrrolidin-2-yl)(thiazolidin-3-yl)methanone;
((2S,4S)-4-(4-(3-D₃-methyl-1-phenyl-1H-pyrazol-5-yl)piperazin-1-yl)pyrrolidin-2-yl)(thiazolidin-3-yl)methanone;
((2S,4S)-4-D-4-(4-(1-(4-D-phenyl)-3-methyl-1H-pyrazol-5-yl)piperazin-1-yl)pyrrolidin-2-yl)(thiazolidin-3-yl)methanone;
((2S,4S)-4-(4-(1-(4-D-phenyl)-3-methyl-1H-pyrazol-5-yl)piperazin-1-yl)pyrrolidin-2-yl)(2,2,4,4-D₄-thiazolidin-3-yl)methanone; and
((2S,4S)-4-(4-(3-D₃-methyl-1-(4-D-phenyl)-1H-pyrazol-5-yl)piperazin-1-yl)pyrrolidin-2-yl)(thiazolidin-3-yl)methanone.

8. A composition comprising a compound of claim 1, or a stereoisomer, tautomer, prodrug, or pharmaceutically acceptable salt or solvate thereof.

9. The composition of claim 8, further comprising a pharmaceutically acceptable carrier.

10. The composition of claim 8, further comprising one or more additional compounds having anti-hyperglycemia activity.

11. The composition of claim 10, wherein at least one of the one or more additional compounds is effective to inhibit the activity of enzyme DPPIV.

12. The composition of claim 11, further comprising a pharmaceutically acceptable carrier.

13. A method of treating diabetes mellitus in a patient, comprising administering to the patient a therapeutically effective amount of a compound of claim 1, or a stereoisomer, tautomer, prodrug, or pharmaceutically acceptable salt or solvate thereof.

14. The method of claim 13, further comprising administering one or more additional compounds having anti-hyperglycemia activity to the patient.

15. The method of claim 14, wherein the at least one or more additional compounds are administered prior to, after, or simultaneously with the compound of Formula (I).

16. The method of claim 14, wherein at least one of the one or more additional compounds is effective to inhibit the activity of an enzyme DPPIV.

17. The method of claim 13, wherein the diabetes mellitus is type II diabetes.

18. (canceled)

19. (canceled)

20. (canceled)

21. The method of claim 13, wherein the compound is selected from the group consisting of:
((2S,4S)-4-(4-(3-methyl-1-phenyl-1H-pyrazol-5-yl)piperazin-1-yl)pyrrolidin-2-yl)(2,2,4,4-D₄-thiazolidin-3-yl)methanone;
((2S,4S)-4-(2,2,3,3,5,5,6,6-D₈-4-(3-methyl-1-phenyl-1H-pyrazol-5-yl)piperazin-1-yl)pyrrolidin-2-yl)(2,2,4,4-D₄-thiazolidin-3-yl)methanone;
((2S,4S)-4-(2,2,3,3,5,5,6,6-D₈-4-(3-methyl-1-phenyl-1H-pyrazol-5-yl)piperazin-1-yl)pyrrolidin-2-yl)(thiazolidin-3-yl)methanone;
((2S,4S)-4-(4-(1-(4-D-phenyl)-3-methyl-1H-pyrazol-5-yl)piperazin-1-yl)pyrrolidin-2-yl)(thiazolidin-3-yl) methanone;
((2S,4S)-4-D-4-(3-methyl-1-phenyl-1H-pyrazol-5-yl)piperazin-1-yl)pyrrolidin-2-yl)(thiazolidin-3-yl)methanone;
((2S,4S)-4-(3,3,5,5-D₄-4-(3-methyl-1-phenyl-1H-pyrazol-5-yl)piperazin-1-yl)pyrrolidin-2-yl)(thiazolidin-3-yl)methanone;
((2S,4S)-5,5-D₂-4-(4-(3-methyl-1-phenyl-1H-pyrazol-5-yl)piperazin-1-yl)pyrrolidin-2-yl)(thiazolidin-3-yl)methanone;
((2S,4S)-4-(2,2,6,6-D₄-4-(3-methyl-1-phenyl-1H-pyrazol-5-yl)piperazin-1-yl)pyrrolidin-2-yl)(thiazolidin-3-yl)methanone;
((2S,4S)-4-(4-(3-D₃-methyl-1-phenyl-1H-pyrazol-5-yl)piperazin-1-yl)pyrrolidin-2-yl)(thiazolidin-3-yl)methanone;
((2S,4S)-4-D-4-(4-(1-(4-D-phenyl)-3-methyl-1H-pyrazol-5-yl)piperazin-1-yl)pyrrolidin-2-yl)(thiazolidin-3-yl)methanone;
((2S,4S)-4-(4-(1-(4-D-phenyl)-3-methyl-1H-pyrazol-5-yl)piperazin-1-yl)pyrrolidin-2-yl)(2,2,4,4-D₄-thiazolidin-3-yl)methanone; and
((2S,4S)-4-(4-(3-D₃-methyl-1-(4-D-phenyl)-1H-pyrazol-5-yl)piperazin-1-yl)pyrrolidin-2-yl)(thiazolidin-3-yl)methanone.

22. The composition of claim 8, wherein the compound is selected from the group consisting of:
((2S,4S)-4-(4-(3-methyl-1-phenyl-1H-pyrazol-5-yl)piperazin-1-yl)pyrrolidin-2-yl)(2,2,4,4-D₄-thiazolidin-3-yl)methanone;
((2S,4S)-4-(2,2,3,3,5,5,6,6-D₈-4-(3-methyl-1-phenyl-1H-pyrazol-5-yl)piperazin-1-yl)pyrrolidin-2-yl)(2,2,4,4-D₄-thiazolidin-3-yl)methanone;
((2S,4S)-4-(2,2,3,3,5,5,6,6-D₈-4-(3-methyl-1-phenyl-1H-pyrazol-5-yl)piperazin-1-yl)pyrrolidin-2-yl)(thiazolidin-3-yl)methanone;
((2S,4S)-4-(4-(1-(4-D-phenyl)-3-methyl-1H-pyrazol-5-yl)piperazin-1-yl)pyrrolidin-2-yl)(thiazolidin-3-yl)methanone;
((2S,4S)-4-D-4-(3-methyl-1-phenyl-1H-pyrazol-5-yl)piperazin-1-yl)pyrrolidin-2-yl)(thiazolidin-3-yl)methanone;
((2S,4S)-4-(3,3,5,5-D₄-4-(3-methyl-1-phenyl-1H-pyrazol-5-yl)piperazin-1-yl)pyrrolidin-2-yl)(thiazolidin-3-yl)methanone;
((2S,4S)-5,5-D₂-4-(4-(3-methyl-1-phenyl-1H-pyrazol-5-yl)piperazin-1-yl)pyrrolidin-2-yl)(thiazolidin-3-yl)methanone;
((2S,4S)-4-(2,2,6,6-D₄-4-(3-methyl-1-phenyl-1H-pyrazol-5-yl)piperazin-1-yl)pyrrolidin-2-yl)(thiazolidin-3-yl)methanone;
((2S,4S)-4-(4-(3-D₃-methyl-1-phenyl-1H-pyrazol-5-yl)piperazin-1-yl)pyrrolidin-2-yl)(thiazolidin-3-yl)methanone;
((2S,4S)-4-D-4-(4-(1-(4-D-phenyl)-3-methyl-1H-pyrazol-5-yl)piperazin-1-yl)pyrrolidin-2-yl)(thiazolidin-3-yl)methanone;
((2S,4S)-4-(4-(1-(4-D-phenyl)-3-methyl-1H-pyrazol-5-yl)piperazin-1-yl)pyrrolidin-2-yl)(2,2,4,4-D₄-thiazolidin-3-yl)methanone; and
((2S,4S)-4-(4-(3-D₃-methyl-1-(4-D-phenyl)-1H-pyrazol-5-yl)piperazin-1-yl)pyrrolidin-2-yl)(thiazolidin-3-yl)methanone.

23. The compound of claim 1, or a stereoisomer, tautomer, prodrug, or pharmaceutically acceptable salt or solvate thereof, selected from the group consisting of:
((2S,4S)-4-(4-(3-Methyl-1-phenyl-1H-pyrazol-5-yl)piperazin-1-yl)pyrrolidin-2-yl)(2,2,4,4-D₄-thiazolidin-3-yl)methanone;
((2S,4S)-4-(4-(1-(4-D-Phenyl)-3-methyl-1H-pyrazol-5-yl)piperazin-1-yl)pyrrolidin-2-yl)(thiazolidin-3-yl)methanone; and
((2S,4S)-4-D-4-(3-Methyl-1-phenyl-1H-pyrazol-5-yl)piperazin-1-yl)pyrrolidin-2-yl)(thiazolidin-3-yl)methanone.
